(19) United States
(12) Patent Application Publication
(10) Pub. No.: US 2006/0149154 A1
(43) Pub. Date: Jul. 6, 2006

(54) Method and apparatus for measuring tissue perfusion

(76) Inventors: Frederick Richard Neason Stephens, New South Wales (AU); Scott Kesteven, New South Wales (AU); Peter Harris, New South Wales (AU)

Correspondence Address: HOGAN & HARTSON LLP;IP GROUP, COLUMBIA SQUARE, 555 THIRTEENTH STREET, N.W., WASHINGTON, DC, 20004, US

(21) Appl. No.: 10/531,600

(22) PCT Filed: Oct. 17, 2003
(86) PCT No.: PCT/AU03/01379

§ 371 Date: Dec. 19, 2005

(30) Foreign Application Priority Data
Oct. 17, 2002 (AU) 2002952144

(51) Int. Cl. A61B 5/02

(52) U.S. Cl. 600/504

(57) ABSTRACT

Apparatus for measuring microcirculatory flow of a target tissue without the necessity for direct contact of a probe is disclosed. The apparatus includes a probe (10) arranged to generate a pulsed source of infrared light (16) and a matched infrared sensor (18) which transduces variations in the reflected light to an electric signal and a signal processor which compares the signal at a first time when the pulsed light source is on with a second time when the pulsed light is off. The signal is processed to reduce or ameliorate the effect of the ambient light in the signal and the Tissue Perfusion Index (TPI) is then calculated. Without the need to contact tissue, the apparatus can be used to measure the TPI for chronic ulcers on the extremities, the surface of the retina, the vascular pulp within a tooth or the surface of internal organs accessed by fiber optic or endoscopic means.

### FIELD OF THE INVENTION

This invention relates to monitoring and diagnostic apparatus and in particular to a method and apparatus for measuring tissue perfusion and analysing blood flow changes as they occur in tissue perfusion.

### BACKGROUND OF THE INVENTION

The blood circulation is divided into two principal divisions. Firstly, the macrocirculation comprises the heart pump and peripheral arteries and veins for distribution of blood to and from the body tissues. Secondly, the microcirculation is a network system of small blood vessels and capillaries. Tissue Perfusion is blood flow through the microcirculation and tissue perfusion determines the viability of body tissues. Changes in microcirculation occur very early in the train of events leading to evidence of circulatory disturbance.

The microcirculation is an interface system between the terminal ramifications of the arterial and venous compartments of the vascular conduit system, the “Macro circulation”. Non-invasive cardiovascular monitoring systems currently in widespread clinical application measure macro parameters such as blood pressure, pulse rate, the Electrocardiogram (ECG) and tissue oxygen saturation (TOS %) which cannot react to early falls in capillary blood flow. While macro parameters provide important feedback to the clinician, they do not reflect the vital activity of the highly sensitive microcirculation.

FIG. 1 illustrates the relationship between the skin microcirculation and deeper vasculature. The invention non-invasively measures the change in the superficial layer of capillary blood flow, at the very interface between the arterial and venous compartments of vascular system.

The human body has a wide variety of cardiovascular, respiratory and basic metabolic reflex mechanisms which endeavour to maintain constancy of blood supply to the organs. Because of the expendability of skin perfusion relative to the vital central organs such as the heart and brain, in the presence of cardiovascular threat, skin microcirculation provides a reserve blood supply by an early compensatory vascoconstrictive mechanism.

Monitoring macro-parameters alone has the following disadvantages. Macro-parameters are insensitive to changes in microcirculation prior to compensatory failure, which determine tissue perfusion. By contrast, by monitoring the skin microcirculation, the clinician is able to observe the start of this compensatory activity to maintain blood supply to the vital organs, and so therefore gains much earlier warning of any impending threat to physiological status.

The macroparameters do not provide information that is specific to an area of interest (such as the border of a skin lesion or wound). By contrast, assessing the microcirculatory flow of a particular tissue provides direct confirmation that the targeted tissue is receiving nutrients and able to remove waste products. Furthermore, the microcirculatory flow of a targeted area e.g. after trauma or grafting can be compared with anatomical counterpart reference areas of tissue.

U.S. Pat. No. 3,796,214 to F. R. N. Stephens discloses a monitoring system, known as the Stephens Tissue Perfusion Monitor or “STPM”, which assesses microcirculatory blood flow in the capillary beds and U.S. Pat. No. 4,442,845 also to F. R. N. Stephens discloses a means of analysing the resulting signal curves. The entire contents of both specifications are incorporated herein by reference.

The STPM's basic parameter, the Tissue Perfusion Index (TPI) is typically derived from the microcirculation of skin or mucous membrane. A non-invasive probe provides a source of light and a matched sensor which transduces the variations in reflected light from the capillary bed into an electrical signal (called the signal pulse curve). The TPI is the short term average running product of a value for the area under the pulse curve and an immediate value for pulse rate per minute. As a result, the TPI provides a continuous quantitative measure of proportional changes, as they occur in blood flow through an observed capillary bed of tissue microcirculation, relative to an initial reference level of tissue perfusion.

Ongoing experience with the STPM has shown it invaluable for warning the clinician of subclinical trends in skin tissue perfusion which could threaten patient wellbeing. For example, steadily declining microcirculation from blood loss during surgery causing fall in TPI and no change in TOS, if uncorrected, can precede clinical shock. Unexpected surgical death occurs because of inability to maintain tissue perfusion. In cardiac shock disturbance of skin capillary circulation is observed and continuous surveillance of skin tissue perfusion, with TPI, provides a vital means of identifying trends in response to treatment.

The pathophysiological state of tissue cannot be assessed from macroparameters such as tissue oxygen saturation, pulse rate or blood pressure. This can be readily demonstrated using staged occlusion of the brachial artery with a sphygmomanometer cuff, where it has been reproducibly observed that up to approximately 90% of capillary bed can close down before significant change occurs in tissue oxygen saturation (refer example data in FIG. 11). In clinical application, it can therefore be appreciated that the parameters of tissue oxygen saturation, blood pressure, pulse and ECG, though important, cannot measure the early vital capillary flow changes of tissue perfusion which signal imminent shock. This is ordinarily because of the physiologically necessary, large capillary reserve.

However, although the STPM and the TPI have been known for many years and their advantages and benefits understood by many in the medical profession, widespread use of the STPM and the TPI has not occurred. This may be due to difficulties in using the apparatus and in particular, the need to have the probe of the monitor in contact with the area being monitored which is undesirable from the point of view of infection risks and when monitoring damaged tissue.

The present invention seeks to address to problems of the prior art and provide an improved method and apparatus for measuring tissue perfusion.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia before the priority date of each claim of this application.

### SUMMARY OF THE INVENTION

In a broad aspect the present invention is directed to a method and apparatus, using a pulsed light source, for measuring microcirculatory flow of a target tissue without the necessity for direct contact of a probe.

According to a first aspect of the present invention there is provided an apparatus for monitoring tissue perfusion including:—

a probe, arranged to generate a pulsed source of infrared light, or light of other spectral wavelength and a matched infrared sensor, or sensor of other suitably matched peak response wavelength, which transduces variations in the reflected light to an electric signal which undergoes signal processing; and,

a signal processor, which receives the electric signal and compares the signal at a first time when the pulsed light source is on with a second time when the pulsed light is off, the first and second times being almost concurrent, and processes the signal to reduce or ameliorate the effect of the ambient light in the signal.

By comparing the signal obtained at these two points in time significant gains in signal to noise ratio can be obtained. Typically the processor digitally samples the signal and analyses it to calculate the Tissue Perfusion Index, as well as other measurements relating to the waveform.

The key advantage of the invention, described in this application, is that by using a pulsed light source and compensating for the background signal or noise due to ambient light; measurement of microcirculatory flow can now be obtained without contact between the probe and the target tissue. This reduces the risk of contact artifact at the areas of microcirculation being analysed. Furthermore, because the probe need no longer contact the target tissue, the use of the apparatus is extended (for example to, chronic ulcers on the extremities, the surface of the retina, the vascular pulp within a tooth or the surface of internal organs, accessed by fiberoptic or endoscopic means). Finally it can now provide more exact and simpler targeting of accessible tissue for analysis of tissue perfusion (for example, angiogenesis at the border of skin grafts, burns or comparison of microcirculatory activity in or around various skin lesions).

Typically the apparatus will further include a display and/or warning system which at the user's discretion, displays either individual waveforms or selected combinations of waveforms, or a continuous single waveform with a running trace of the TPI trend. The system may be arranged so that selected characteristics of the waveform shape and/or changes in the TPI can activate an audible alarm when the measurement moves above or below pre-defined limits.

The light may or may not be monochromatic.

In a related aspect the present invention provides a method for measuring microcirculatory blood flow in a body comprising the steps of:

using an emitter of pulsed light to irradiate an area of the body for measurement of microcirculatory changes;

receiving light reflected from the area at a distance from the area being irradiated by the incident light; and

determining from the reflected light a measure of the changes that correspond with the pulsatile filling and partial emptying of the microcirculation.

The method will further include the step of calculating the Tissue Perfusion Index and displaying key signal characteristics of said index.

### BRIEF DESCRIPTION OF THE DRAWINGS

Specific examples of the present invention will now be described, by way of example only, and with reference to the accompanying drawings in which:—

FIG. 1 shows the dermal vasculature; by courtesy of Waverly Publishers-Williams & Williams Wilkins

FIG. 2 is a graphic illustration of a signal derived from a probe embodying the resent invention;

FIG. 3a is a schematic drawing of a first embodiment of a probe

FIG. 3b is an enlarged end view of the probe of FIG. 3a;

FIG. 4 is a schematic drawing of a second embodiment of a probe;

FIG. 5 is a schematic drawing of a third embodiment of a probe;

FIG. 6 is a schematic drawing of a fourth embodiment of a probe;

FIG. 7 is a schematic drawing of a fifth embodiment of a probe;

FIG. 8 is a schematic illustration of signal acquisition steps of a system embodying the present invention;

FIG. 9 is a schematic illustration of the signal processing steps of a system embodying the present invention;

FIG. 10 is a graph illustrating emitter and sensor voltages;

FIG. 11 illustrates sample readings of Tissue Perfusion Index (PI) compared with Tissue Oxygen saturation (TOS), at skin of forearm and finger, during staged occlusion of the brachial artery with a sphygmomanometer, with a fall of almost 90% in TPI occurring before significant change is TOS;

FIG. 12 illustrates sample readings from the use of a stand off probe embodying the present invention to check blood supply to the scalp, during staged occlusion of the left and right carotid arteries in turn.

FIG. 13 illustrates a TPI display of a patient asleep;

FIG. 14 illustrates a TPI display of the patient of FIG. 13 wakening; and

FIG. 15 shows a slow pulse curve using a standoff probe targeting scalp skin of a hypertensive subject with Bradycardia on a beta blocker drug (Atenolol).

FIG. 16 shows a TPI trend curve illustrating an example of rapid intravenous administration of less than 200 ml of normal saline solution.

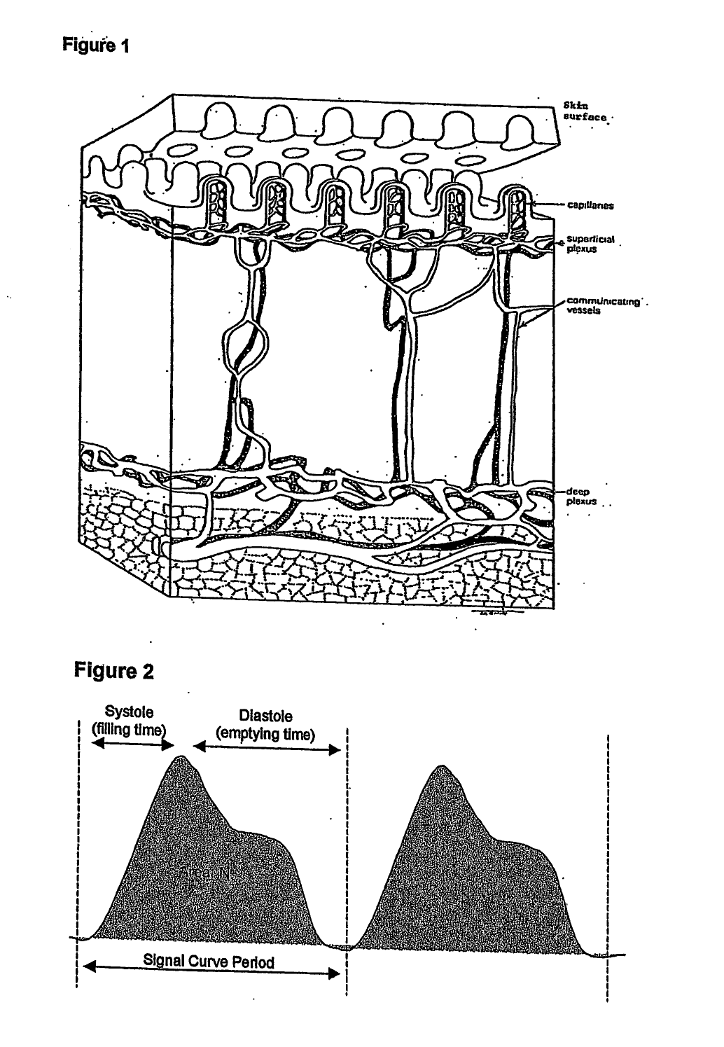

### DETAILED DESCRIPTION OF PREFERRED EMBODIMENTS

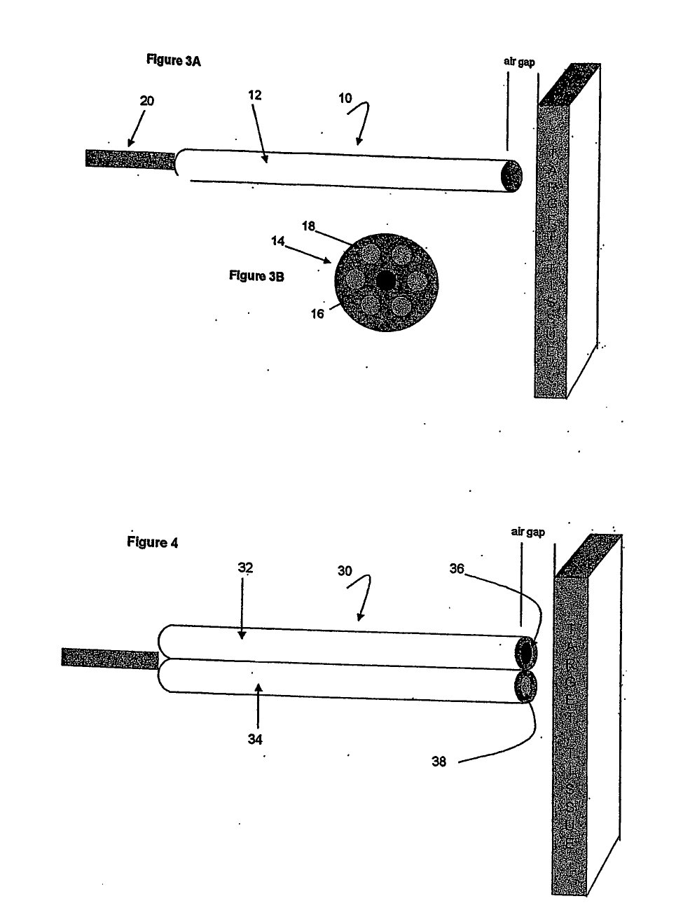

Referring to the drawings FIGS. 3a and 3b shows a first design of a probe 10 embodying the present invention. The probe 10 comprises a high density, black polyethylene tube 12 which is 7 mm in diameter and 105 mm long which includes a light emitting and a light sensing element, 14 which as is best seen in FIG. 3b is a circular, compounded light emitting and sensing device and is placed at the end of the tube. The element 14 comprises a central emitter 16 and an array of surrounding sensors 18. The emitter emits a pulsed light source. An electrostatically shielded cable 20 transfers the electrical signal from the probe 10 to signal processing electronics. Depending on the application, the number of sensors or more than one emitter may be used. For example, in FIG. 3B, an alternate probe design may comprise a central sensor with one or more emitters.

The principal of operation of the system of the present invention is as follows.

The absorption of light entering a tissue can be said to follow the Beer-Lambert law of attenuation. Consequently, any backscattered light that reaches the sensors 18 is derived primarily from that region of tissue closest to the sensor.

The time varying signal is generated by absorptance levels of the incident infrared light from the probe which falls on the observed tissue's microcirculation during the filling and partial emptying of the microcirculation with blood at each heart beat. The peak wavelength response of the emitter and sensor are approximately matched and include the isobestic point (805 nm) on the absorption curves of oxygenated and deoxygentated blood. Importantly, the extravascular interstitial tissue enmeshing the microcirculation is relatively non-absorbent of light at this wavelength in comparison to the pulsatile blood flow of the capillary bed. This means that the backscattered light changes markedly in response to the pulsatile changes in the microcirculation.

As the microcirculation is filled during systole, light absorption increases and light back-scattered to the probe falls. The system circuitry records this fall in backscattered light as indicative of more red blood cells being present in the observed field and proportionally increases the probe's signal. Conversely, as the microcirculation empties during diastole, absorption decreases (and so backscattered light increases), and the probe signal level falls. Consequently, the degree to which the signal rises and falls is closely related to the pulsatile volume of red blood cells passing through the observed field at any instant. This resulting signal is integrated over each heart beat (corresponding to the area under the pulse curve), and multiplied by the heart rate. These products are then averaged over a pre-determined minimum short running time frame to provide an index of tissue perfusion, (that is, the TPI). Put mathematically:

$$TPI varies as \begin{matrix}Curve Area \\ ( average )\end{matrix} \times \begin{matrix}Heart Rate \\ ( average )\end{matrix}$$

Hence in a given running time frame,

$$TPI varies as \frac{Red Cells}{Cardiac cycles} \times \frac{Cardiac Cycles}{Minute}$$

$$TPI varies as \frac{Red Cells}{Minute}$$

That is, the TPI varies in proportion to any changes in observed capillary blood flow at any given time.

The TPI may be directly expressed as:
TPI=ƒA×HR×k
where:
A=running value for area under signal curve
HR=value for Heart Rate
k=physiological constant for specific tissue

FIG. 2 illustrates the form of a typical time varying signal derived from capillary bed by a probe. FIG. 8 is a schematic diagram which sets out the key functional blocks in the signal acquisition by the system of the present invention.

A pulsed light source is used. The pulsed light source enables data acquisition from a signal relatively free of background artifact (“noise”) due to interference from ambient light. This enables tissue to be observed, either from a stand-off position across an air gap (for example, 30 mm), or using fibre optic bundles to direct a highly focused light source to the target tissue, or provide highly focused sensors to collect light from specific locations. This ability to observe tissue at a distance greatly expands the monitoring capabilities of the new system compared with the existing system and a number of possible novel uses of the system are set out below.

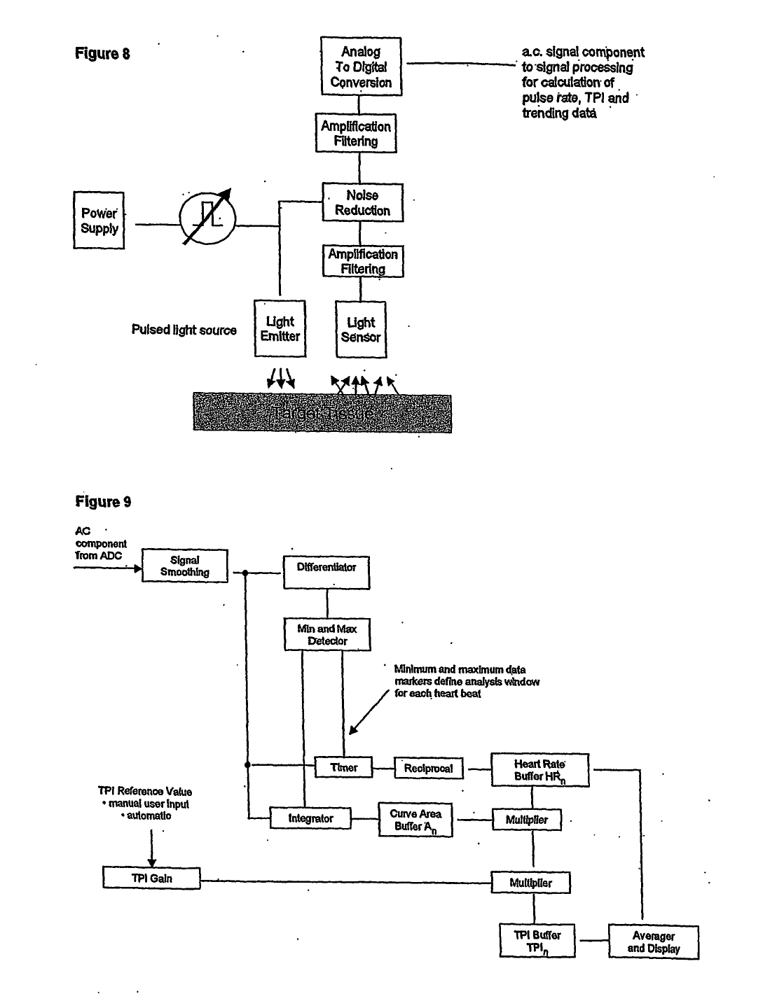

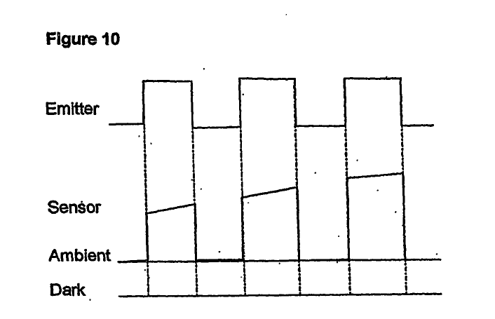

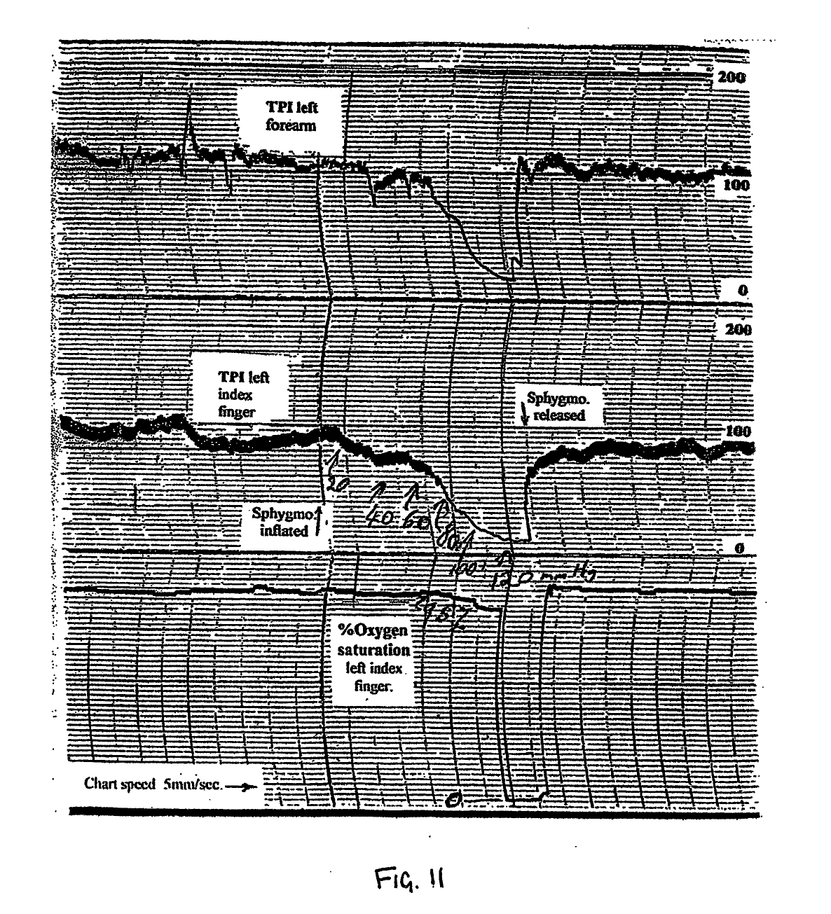

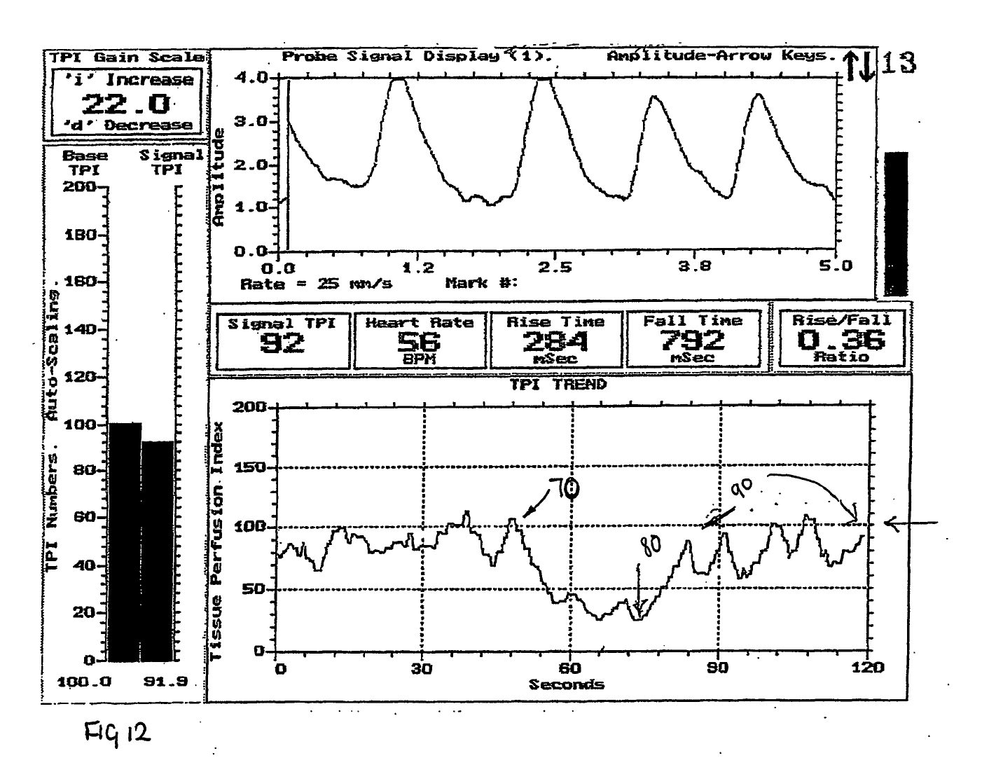

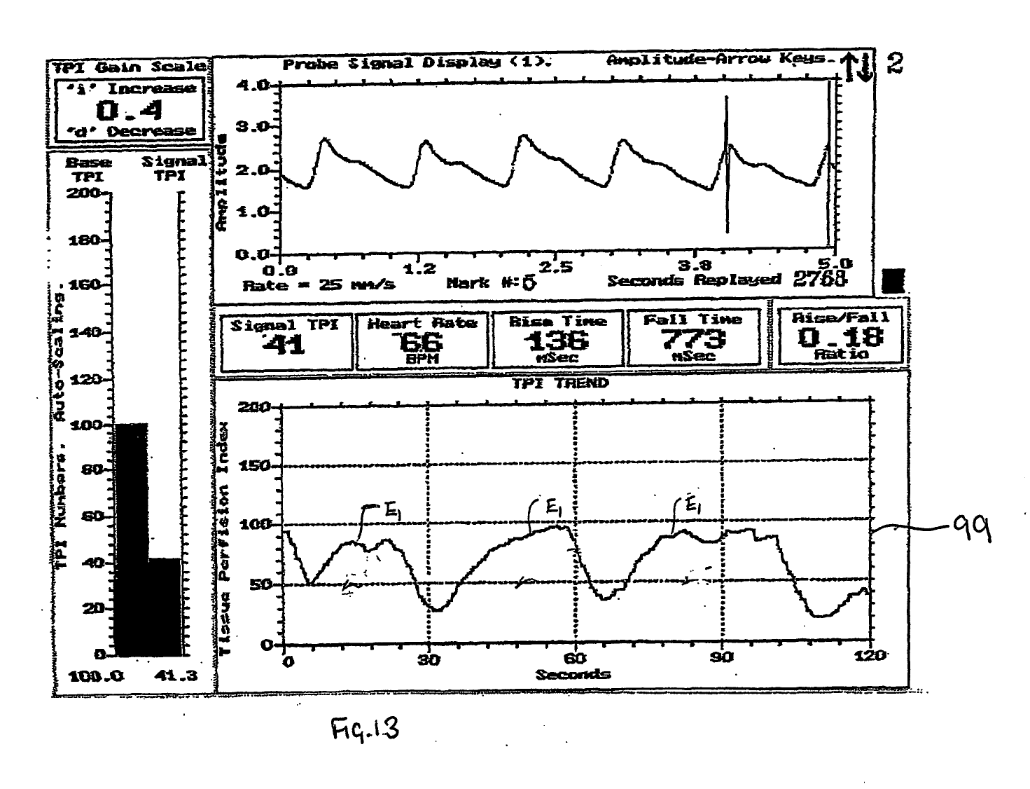

FIG. 9 outlines the key signal processing blocks. The electrical signal from the light sensor undergoes Analog to Digital Conversion and the resulting data stream is then smoothed. Following peak detection of the differentiated data stream by use of an active threshold technique, the times at which maximums and minimums occurred in the data stream are determined. These time points are then used as markers to calculate (i) the heart rate (from the time between two successive minimums) and (ii) the TPI (pulse curve area×HR), during this interval. The resulting data streams are separately buffered, for example, the heart rate buffer acquires six seconds of data, while the TPI buffer acquires three seconds. The TPI is then multiplied by the TPI gain value set either manually or automatically using the current signal level as a reference for subsequent data acquisition. Subsequent TPI values are then compared to this Reference TPI to reflect change in tissue perfusion from an initial state, or tissue perfusion relative to a different location.

In clinical application, the TPI measures change in microcirculation as it occurs from an initial reference level. For example, if the system is being used to monitor a patient during general anaesthesia, the base reference would be established with the patient in an early settled state prior to anaesthesia.

As a second example, if the system is used to assess capillary activity in a target tissue, for example, a site of inflammatory or neoplastic tissue in skin, the reference level would be taken from the adjoining normal skin of the subject comfortably supine.

The shape of the signal curve varies with tissue compliance to flow, as physiological or pathological changes in tissue are encountered and so the time point estimates of TPI signal are also used to calculate other characteristics of the signal curve (for example, the rise time and fall time) which is one characteristic of signal shape. The changes in signal curve shape are expressed as variations in rise time Tᵣ (msec) and fall time T_{f} (msec). These analysis techniques are described in the Prior Art (refer U.S. Pat. Nos. 3,796,214 and 4,442,845), the entire contents of which are incorporated herein by reference.

The system is controlled using a Personal Computer interface, not illustrated. Signal processing and display parameters are controlled using keystrokes and the waveform(s) and signal characteristics are displayed on the computer monitor in real time. These digitised signals may also be optionally logged as a digital file for recording and post-processing.

The PC interface provides a multitude of options of display of the information. For example, if the system is being used during anaesthesia, a declining TPI can indicate compensatory vasoconstriction of skin from blood loss and warning of impending cardiovascular shock. A declining TPI can also indicate clinically non-evident accumulating tissue oedema (for example, from excess intravenous saline osmotically compromising the capillary bed). The clinician is alerted to these otherwise unknown important disturbances by an optional on/off alarm system which sounds if the TPI, calculated as a moving average figure, moves beyond a high or low predefined range for a finite time (for example 8 seconds) from an initial reference level. The changes in tissue perfusion of the targeted organs are identified for the clinician long before macro-parameters such as blood pressure, heart rate or tissue oxygen saturation, all late indicators of disturbance, show any change.

Alternatively, the system's display can be configured to capture and display the TPI at various locations of the targeted tissue to monitor its viability (for example, assessing the return of blood supply to a skin graft or characterising the microcirculation of a skin lesion, or at the border of a skin lesion).

FIG. 4 illustrates a second embodiment of a probe 30 in which two high density polyethylene tubes 32, 34 are located side by side. One tube 32 contains a light emitting device 36 arranged to emit a pulsed light source and the other tube 34 contains a light sensing device 38. An analogous implementation using fibre optic cable could be readily employed to provide much smaller, more flexible probe designs using this approach.

FIG. 5 illustrates yet a further probe design in which a light emitter 40 and a light sensor 42 are mounted side by side close to the end of a tubular probe 44, suitable for the observation of intrauterine and cervical tissue or for intra-rectal examinations.

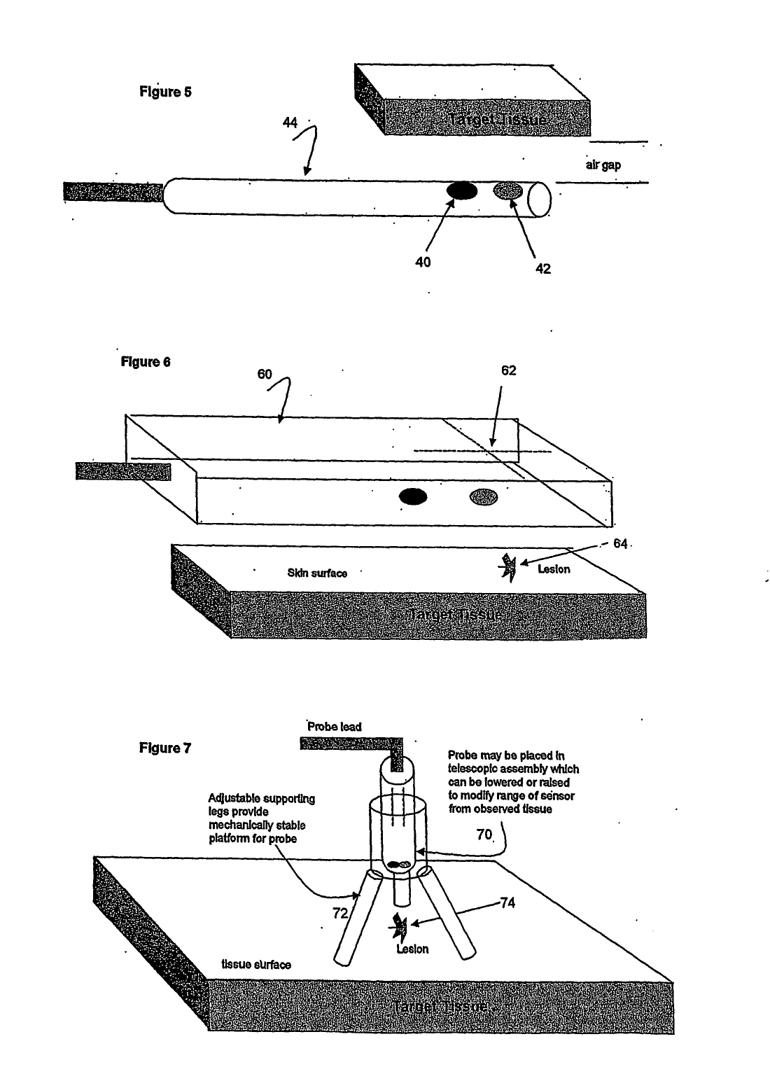

FIG. 6 illustrates yet a further probe 60 which may be transparent and is approximately 20 mm long×15 mm wide×3.5 min deep and can be used as a multi-purpose probe for analysing microcirculation at a point of observation on the skin surface. The back of the probe incorporates marks 62 over the sensor to facilitate alignment. The skin may be marked to enable alignment of the sensor over the targeted area of tissue 64.

FIG. 7 illustrates yet a further probe 70 which is mounted on adjustable legs 72 to facilitate placement. The optical elements of the probe may be mounted in a telescopic tube to enable different areas of tissue to be examined, such as a skin lesion 74.

In basic application the previous system described in earlier prior art has been invaluable for detection of autonomic disturbances such as due to lightness of anaesthesia, or for correction at skin level of a trend to preshock and for accurate blood replacement following blood loss. However, the invention described herein incorporating a pulsed light source greatly expands the monitoring capabilities to enable assessments of important tissue viability in previously difficult to access areas.

Such areas may include:

observations of damaged tissue in burns units,

variations in re-vascularisation of tissue in trauma units and in the field of dermatology or following skin grafting, or in the management of post-operative wound breakdown,

assessment of retinal microcirculation by splitting and processing back reflected light from a light beam in a slit lamp optical instrument,

assessment of viability of tooth pulp tissue through the enamel of the crown of the tooth, and

the use of two way fibre optic bundles allows viability in difficult to access organ tissues to be monitored, eg, through a ureter to the pelvis of a transplanted kidney.

The assessment of TPI trend in observed microcirculation can provide characteristic waveforms in the TPI trend display that can be triggered by various central nervous system status changes (for example, in the state of sleep or from transient falls in cerebral blood flow) or autonomic status change (for example, such as from afferent stimuli caused by a distending bladder).

In yet another application, arterial stenoses may be located by observing the changes in the TPI reading of skin during sequential occlusion of each of the arterial supply vessels by direct pressure. In this particular application, the TPI is a diagnostically valuable supplement to other vascular diagnostic methods (e.g. Ultrasound Doppler systems).

In the field of neurology responses in microcirculation occur from influences such as from sympathetic blockade, reflex sympathy dystrophy and causalgia which by tissue blood flow activity and signal curve analysis can be accurately observed and recorded.

### EXAMPLES

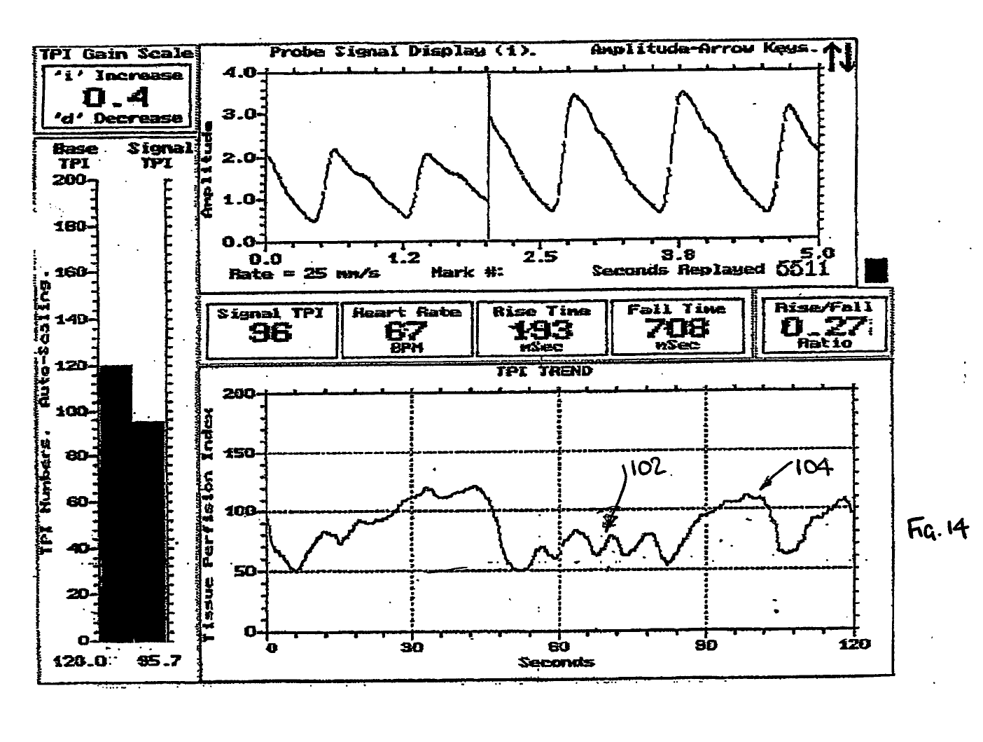

FIG. 12 illustrates sample readings from the use of a stand off probe embodying the present invention to check blood supply to the scalp. The point of observation of the skim was over an air gap of over 10 mm and under bright fluorescent lighting. The subject's left and right carotid arteries were pressure occluded in turn. The results clearly show a blood supply problem with the left carotid artery supply because compression of the right carotid at 70 produced an excessive 75% fall in scalp tissue perfusion index and an unpleasant near loss of consciousness for the subject who became quickly aware of a passing out sensation. That compares with a smaller 20% fall in TPI and no subject response when the left carotid artery was compressed. When the right carotid artery was released at 80 the TPI returned to normal the base reference TPI level being 100.

The TPI signal also showed “Entrainment Waves” or “E-Waves” at 90. It is known that certain body systems have their own particular respective oscillatory frequency states. Both the relatively slow respiratory rate and the faster beating heart rate can vary promptly. These characteristic oscillatory frequency states differ widely. For example, physical exertion, sudden emotional stress, the state of sleep, walking from sleep and postural rearrangements such as raising ones body to a standing position from a supine position causes transient disturbance to the existing dynamics of blood flow in the body.

Whilst the display of tissue perfusion index in the system of the present invention, clearly shows quantified changes in capillary flow, the trend display can also show wave forms with particular characteristic period changes which appear to result from interaction of multi-factorial influences. These changes are referred to as entrainment wave responses or E-Waves. It has been demonstrated that frequencies lower than heart rate, exist in the cardio vascular system (see Traube, Hering and Mayer (Periodic posture stimulation of baroreceptor and local vasomotor reflexes, J. Biomed. Eng. 1992, Vol. 14, July)). It was found that two frequencies were present, one corresponding to breathing rate of about 4 to 6 seconds and another with a period of about 10 seconds, the latter thought to be due to blood pressure control mechanism. This 10 second frequency was called the THM wave after its discovers. However, until the apparatus of the present invention was developed, these wave forms have not been readily observable.

Both the THM waves period of about 10 seconds and the shorter respiratory related waves with a period of about 4 to 6 seconds, show clearly when present in the continuous two minute TPI trend trace of the computerised monitor. However, during state of sleep, as shown in the TPI display 99 of FIG. 13, E wave forms E₁ of a period of around 20-30 seconds, occur not infrequently. With arousal of the subject, these longer period waveforms spontaneously shortened down to around 10 seconds as shown in FIG. 14 at 102. If the subject drifts back to sleep the E-Waves lengthen again as shown at 104. These observations were recorded during a conducted hospital study.

Slope varying E-Waves of around 60 seconds appear to relate to the bladder filling with urine. The mechanism of these happenings is not yet understood. It is possible that bladder stretch reflexes generate afferent automatic stimuli which go to the mid brain and higher hypothalamic centres and result in changes to dynamics of tissue blood flow. The resultant effect of this is long TPI trend E-Waves. The apparatus of the present invention provides a means to observe, record and explore subclinical activities within the micro circulation to which conventional parameters of BP pulse, ECG and tissue oxygen percentage saturation are insensitive.

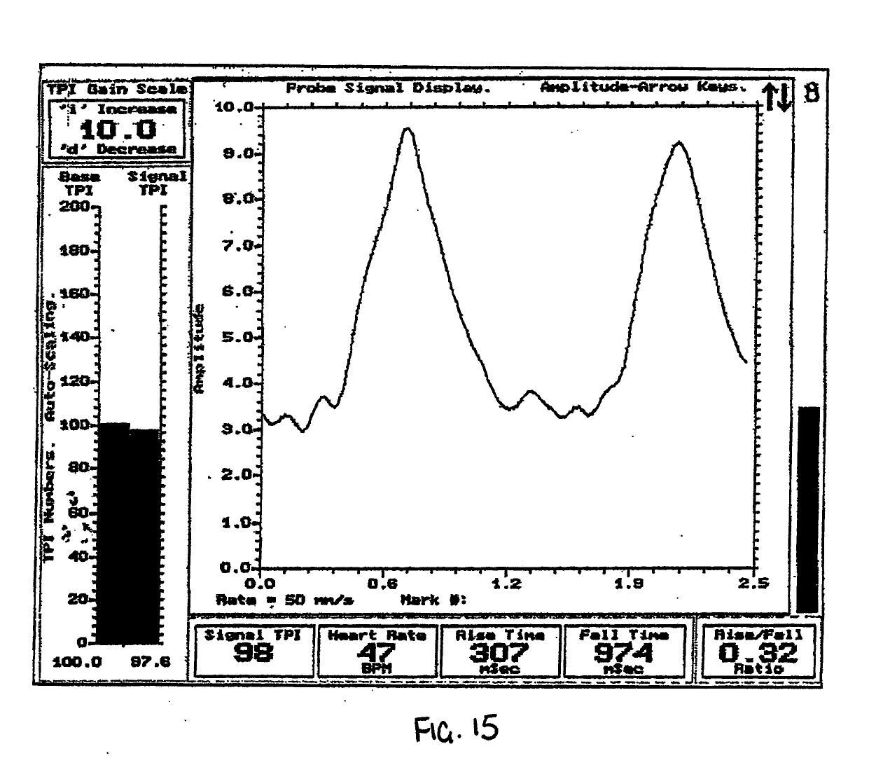

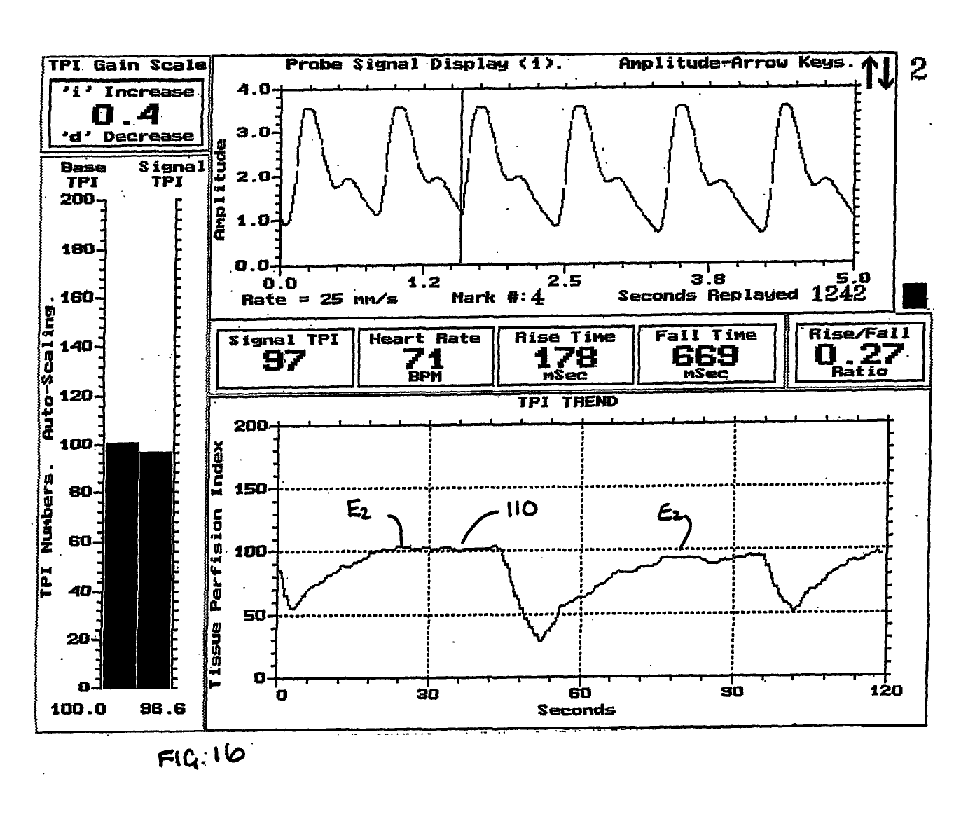

FIG. 15 shows a slow pulse curve using a standoff probe of a hypertensive subject with Bradycardia approximating 50 BPM on medication of atenolol 50 mg once daily. A pause of about 400 milliseconds occurs prior to the start of each systolic capillary film mode. The graph shows the shape of the probe signal display before conversion to the TPI.

FIG. 16 shows a TPI trend curve 110 illustrating an example of rapid intravenous administration of less than 200 ml of normal saline solution in a patient. It has induced E waves E₂ having a 40 to 50 second period seen in the TPI trend curve 110 before any significant change in TPI. This suggests the start of an osmotic disturbance caused by the normal saline solution.

While this application describes one embodiment of the invention, other variations in signal acquisition design to achieve the same capabilities are possible. For example, data may be acquired while the light emitter is switched off, to provide an active sample of background noise, which can then be digitally subtracted from the signal. Band pass filters may be applied to reduce noise outside the relatively low frequencies of concern before data analysis. As noted there is a wide range of probe designs of which several examples are disclosed.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A perfusion monitor for monitoring tissue perfusion in a body including:—
a probe, arranged to generate a pulsed source of light for irradiation onto a part of a body and a matched sensor, which transduces variations in the reflected light to an electric signal; and,
a signal processor, which receives the electric signal and compares the signal at a first time when the pulsed light source is on with a second time when the pulsed light is off, the first and second times being almost concurrent, and processes the signal to reduce or ameliorate the effect of ambient light in the signal.

2. A perfusion monitor for monitoring tissue perfusion in a body as claimed in claim 1 wherein the probe is arranged to generate a pulsed source of infrared light.

3. A perfusion monitor for monitoring tissue perfusion in a body as claimed in claim 1 further including:
means for digitally sampling the signal;
means for generating a pulse curve from the signal means for calculating a heart rate (HR) from the pulse curve;
means for determining a running value A for the area under the pulse curve: and
means for calculating a Tissue Perfusion Index (TPI) defined by:
TPI=A×HR×k
where:
A=running value for area under signal curve
HR=value for Heart Rate
k=physiological constant for specific tissue.

4. A perfusion monitor for monitoring tissue perfusion in a body as claimed in claim 3 further including a display and/or warning system which at the user's discretion, displays either individual waveforms or selected combinations of waveforms, or a continuous single waveform with a running trace of the TPI trend.

5. A perfusion monitor for monitoring tissue perfusion in a body as claimed in claim 4 wherein the warning system is arranged so that selected characteristics of the waveform shape and/or changes in the TPI activate an audible alarm when the measurement moves above or below pre-defined limits.

6. A perfusion monitor for monitoring tissue perfusion in a body as claimed in claim 2 wherein the light generated by the pulsed source is monochromatic.

7. A perfusion monitor for monitoring tissue perfusion in a body including:—
a probe, arranged to generate a pulsed source of light for irradiation onto a part of a body and a matched sensor, which transduces variations in the reflected light to an electric signal; and,
a signal processor, which receives the electric signal and compares the electrical signal at a first time when the pulsed light source is on with a second time when the pulsed light is off, the first and second times being almost concurrent, the signal processor including
means for processing the electrical signal to reduce or ameliorate the effect of ambient light in the signal;
means for generating a pulse curve from the signal
means for calculating a heart rate (HR) from the pulse curve;
means for determining a running value A for the area under the pulse curve:
means for calculating a tissue perfusion index (TPI) from HR×pulse curve area, where
TPI=A×HR×k
where:
A=running value for area under signal curve
HR=value for Heart Rate
k=physiological constant for specific tissue.

8. A perfusion monitor for monitoring tissue perfusion in a body as claimed in claim 7 wherein the probe includes two fibre optic cable tubes disposed side by side through one of which the pulsed light source is transmitted and through the other of which the reflected light is received.

9. A method of measuring microcirculatory blood flow in a body comprising the steps of:
using an emitter of pulsed light to irradiate an area of the body for measurement of microcirculatory changes;
receiving light reflected from the area at a distance from the area being irradiated by the incident light; and
determining from the reflected light a measure of the changes that correspond with the pulsatile filling and partial emptying of the microcirculation.

10. A method of measuring microcirculatory blood flow in a body as claimed in claim 9 wherein the step of determining from the reflected light a measure of the changes that correspond with the pulsatile filling and partial emptying of the microcirculation includes the steps of
digitally sampling the signal;
generating a pulse curve from the signal calculating a heart rate (HR) from the pulse curve;
determining a running value A for the area under the pulse curve: and
calculating a Tissue Perfusion Index (TPI) defined by:
TPI=A×HR×k
where:
A=running value for area under signal curve
HR=value for Heart Rate
k=physiological constant for specific tissue and displaying key signal characteristics of the calculated TPI index.

11. A method of calculating the tissue perfusion index for an area or part of a body comprising the steps of:
using an emitter of pulsed light to irradiate an area of the body part for measurement of microcirculatory changes;
receiving light reflected from the area at a distance from the area being irradiated by the incident light;
digitally sampling the signal;
generating a pulse curve from the signal calculating a heart rate (HR) from the pulse curve;
determining a running value A for the area under the pulse curve: and calculating the Tissue Perfusion Index (TPI) defined by:
TPI=A×HR×k
where:
A=running value for area under signal curve
HR=value for Heart Rate
k=physiological constant for specific tissue.

12. (canceled)

13. (canceled)
